(19) United States
(12) Patent Application Publication
(10) Pub. No.: US 2026/0207361 A1
(43) Pub. Date: Jul. 23, 2026

(54) LOADING SYSTEM FOR A STENT DEVICE

(71) Applicant: T-Heart SAS, Paris (FR)

(72) Inventor: Ninon Roux, Paris (FR)

(21) Appl. No.: 19/140,865

(22) PCT Filed: Dec. 14, 2023
(86) PCT No.: PCT/EP2023/085980

§ 371 Date: Jun. 18, 2025

(30) Foreign Application Priority Data
Dec. 20, 2022 (WO) PCT/EP2022/087080

(51) Int. Cl. A61F 2/95; A61F 2/24

(52) U.S. Cl. CPC A61F 2/9525; A61F 2/2418

(57) ABSTRACT

The present invention pertains to the field of loading systems for a stent device for a prosthetic heart valve, in particular for tricuspid or mitral prosthetic heart valves, for providing the stent device, preferably comprising a prosthetic heart valve, in a predefined collapsed state, as may be required for deployment and/or use with a catheter. Accordingly, a loading system for collapsing a stent device for a prosthetic heart valve into a predefined state is suggested, comprising a guiding device (10) and a first biasing device (22). The guiding device (10) comprises an open cavity (16) defined by a wall and configured to receive a longitudinal end portion of the stent device, wherein a diameter of the cavity (16) increases from a longitudinal end of the cavity (16) defining an opening (18) to an opposing longitudinal end of the cavity (16), and a first cylindrical portion (12) defining a through-hole having a predefined inner diameter and extending from said opening (18) in a longitudinal direction away from the cavity (16). The first biasing device (22) is configured to accommodate the first cylindrical portion (12) and comprises a coupling mechanism (34) for coupling the first biasing device (22) to the stent device received in the cavity (16) and a biasing mechanism, wherein the biasing mechanism is configured to bias a portion of the stent device into the through-hole via the cavity (16) and the opening (18) in the coupled state of the stent device.

### CROSS-REFERENCE TO RELATED APPLICATIONS

This application is the U.S. National Stage of PCT/EP2023/085980, filed on Dec. 14, 2023, which claims priority to PCT/EP2022/087080, filed on Dec. 20, 2022, the entire contents of which are incorporated herein by reference.

### TECHNICAL FIELD

The present disclosure relates to the field of loading systems for a stent device, particularly a stent device comprising a prosthetic heart valve, in particular a prosthetic tricuspid or mitral heart valve, for providing the stent device in a predefined collapsed state, as may be required e.g. for deployment and/or use with a catheter.

### BACKGROUND

Dysfunctional heart valves may lead e.g. to valve regurgitation, which is a common medical problem and is associated with significant challenges. For example, patients suffering from tricuspid valve regurgitation generally suffer from a chronically dysfunctional fluid retention and have a low cardiac output. In order to improve functionality of the valve, replacement techniques of defective heart valves, in particular by a percutaneous route or by a minimally invasive route, have been established.

Such replacement techniques require that a prosthetic heart valve or stent device thereof is introduced to the target site in a collapsed state prior to deployment at the appropriate level. The stent device comprising the prosthetic heart valve is then subsequently allowed to gradually expand. In the (fully) collapsed state, the stent device comprising the prosthetic heart valve may be loaded in a delivery system, such as a catheter, wherein one or more sheaths may cover the stent device with the prosthetic heart valve or portions thereof during introduction into the patient and prior to deployment at the target site. The collapsed state thereby facilitates or enables the introduction into the patient in accordance with the anatomical constraints.

Recent developments in the design of stent devices with a prosthetic heart valve have suggested variations in the radial extension of support or anchoring arms and/or an increase in the overall dimensions of the stent device, in particular in view of stent device with prosthetic tricuspid valve designs. Due to the native valve's complexity and the pathological anatomical landscape, stent devices with prosthetic heart valve may furthermore comprise larger longitudinal extensions, so as to provide cross-valve extensions, e.g. atrioventricular extensions spanning between a ventricular and atrial portion. To improve adaptability to the anatomical landscape, e.g. the annulus of an atrioventricular valve, various circumferential extensions have been suggested.

The improvements of stent devices with prosthetic heart valves in terms of their adaptability to the native anatomical landscape, however, have been found to raise issues when loading the stent device into a collapsed state. In particular, increased and/or different radial biasing forces may be individually required for particular portions of the stent device. Moreover, the risk of intertwining components, which impair proper loading, may be increased. State of the art loading approaches that are based on a single collapsing step and/or designed for stent devices with prosthetic heart valves with a substantially cylindrical design, e.g. having only central stabilizers or supports, may not be suitable for stent device with prosthetic heart valves with a more prominent anchoring frame or even multiple anchoring frames.

Accordingly, there is a need for loading systems specifically adapted to stent devices for prosthetic heart valves, which reduce the above problems and which facilitate the collapsing of the respective stent device with the prosthetic heart valve, without significantly modifying the relative arrangement of the various portions.

### SUMMARY

It is an object of the present disclosure to provide a loading system for a stent device for a prosthetic heart valve which abrogates at least some of the above draw-backs for the clinical practice.

Accordingly, a loading system for collapsing a stent device for a prosthetic heart valve into a predefined state is suggested, comprising a guiding device and a first biasing device.

The guiding device comprises an open cavity defined by a wall. The guiding device is configured for receiving a longitudinal end portion of the stent device, wherein a diameter of the cavity increases from a longitudinal end of the cavity defining an opening to an opposing longitudinal end of the cavity. The guiding device furthermore comprises a first cylindrical portion defining a through-hole having a predefined inner diameter and extending from said opening in a longitudinal direction away from the cavity.

The first biasing device is configured for accommodating the first cylindrical portion. It comprises a coupling mechanism for coupling the first biasing device to the stent device received in the cavity and a biasing mechanism. The biasing mechanism is configured for biasing a portion of the stent device into the through-hole (first cylindrical portion) via the cavity and the opening in the coupled state of the stent device.

By increasing diameter and/or increasing cross-sectional area of the cavity in a direction away from the opening, a transition in the diameter is provided, which facilitates a radial biasing of the portion of the stent device being received in the cavity. That is, a radial extension of the stent device may be reduced towards the opening, such that an introduction into the through-hole of the directly adjacent first cylindrical portion may be facilitated upon application of the biasing mechanism.

The biasing into the through-hole is additionally supported by the coupling mechanism, which ensures that the first biasing device and/or its biasing mechanism may exert a biasing force to the received stent device. In embodiments, the coupling mechanism is provided at a longitudinal end portion of the stent device being closest to the opening prior to the biasing. In embodiments, the coupling mechanism and biasing mechanism are configured to exert a tensional force to said end portion of the stent device via the opening and via the first cylindrical portion. In other words, a pulling force is applied to the stent device by the coupling mechanism and the biasing mechanism so as to provide a longitudinal displacement of the stent device towards the opening and the first cylindrical portion of the guiding device. The different diameters of the cavity facilitate the radial biasing required to assume a collapsed state.

Hence, in the assembled state, the stent device is mechanically coupled to the first biasing device in such a manner that the biasing mechanism is in physical communication with the stent device received in the cavity of the guiding device.

In embodiments, the opening of the cavity defines the smallest inner diameter of the cavity and may form a junction between the cavity and the through-hole of the first cylindrical portion. Accordingly, the portion of the stent device is biased radially inward before entering the through-hole of the first cylindrical portion. It may hence be accommodated in a collapsed state in said through-hole by advancing the thus radially biased portion of the stent device into the first cylindrical portion. The radial biasing and the longitudinal displacement via the opening and into the through-hole are facilitated both by the different diameters of the cavity and the biasing and coupling mechanisms.

The stent device may be formed of a deformable material, so as to transition between a collapsed and expanded or deployed state. For example, the stent device may be formed from a metallic memory material, e.g. braided nitinol, such that its shape may be predefined and may be modified, e.g. collapsed, within a predefined temperature range, typically (much) lower than the body temperature of the patient, e.g. between 5° C. and 10° C. The stent device may be defined by a mesh-shape and may e.g. be formed as a lattice or as a plurality of polygonal or ellipsoid cells that are connected either directly to each other or via struts. Thereby, collapsing and expansion of the stent device may be facilitated compared e.g. with solid structures.

According to an embodiment, the loading system is adapted for a stent device comprising a prosthetic heart valve, in particular a prosthetic tricuspid or mitral heart valve. According to a particular embodiment, the stent device comprises a prosthetic heart valve as described in WO 2017/089179 A1 or in FIGS. 9B, 9C, or 11B of WO 2020/234199 A1. According to an embodiment, the loading system is adapted for a stent device comprising (a) a mesh-shaped body extending in an axial direction, wherein the body defines an inner portion (providing a passageway from a proximal end to a distal end of the body) and (b) an outer portion comprising supporting or anchoring frames (also called supporting or anchoring arms), which extend radially outward from the body, i.e. from the distal end of the mesh-shaped body towards the proximal end, wherein each supporting or anchoring frame extends radially outwards, i.e. supporting or anchoring frames may be formed as respective projecting features such as petals extending from the mesh-shaped body.

In embodiments, the wall of the cavity defines an inner guiding surface and/or inner support surface for the stent device configured for facilitating the biasing of the stent device in the coupled state (with coupling mechanism) and upon actuation of the biasing mechanism. Accordingly, a continuous inner surface may be provided between the respective longitudinal ends and/or between the different diameters of the cavity. Thereby, the portion of the stent device received in the cavity may be in contact with the inner surface during loading and collapsing and hence be held. Support of the stent device may ensure that a relative arrangement of different features of the stent device, in embodiments including the prosthetic heart valve, may be maintained during biasing and collapsing of the stent device at least to some extent.

Such a contact may furthermore facilitate the biasing upon longitudinal displacement of the stent device by guiding the portion of the stent device towards the opening, i.e. the smaller diameter of the cavity. The cavity may hence be adapted according to the shape and dimensions of the portion of the stent device received in the cavity and/or to be accommodated in the through-hole of the first cylindrical portion.

In embodiments, to further facilitate the radially inward biasing and longitudinal displacement towards the first cylindrical portion, the diameter of the cavity gradually or continuously increases from the opening towards the opposing longitudinal end of the cavity. Such a gradual (e.g. non-stepwise) or continuous increase of diameter may ensure that contact with the received portion of the stent device is improved during the biasing while longitudinal displacement is not impaired by steps or edges.

In embodiments, the cavity has a conical or parabolic shape in a longitudinal section of the cavity and/or has a rotationally symmetrical shape along a longitudinal axis defined by the cavity. The conical or parabolic shape may provide an improved biasing force distribution and centering of said force towards the opening. The curvature and dimensions may be adapted to the shape and dimensions of the portion of the stent device received in the cavity. For example, the cavity and/or guiding device as a whole may comprise a funnel shape or half egg shape. Accordingly, a contact or support surface may be optimized, thereby reducing the risk of intertwining or twisting of outer features of the stent device and/or further improving the translation of the biasing force to the stent device.

An inner surface of the wall defining the cavity and an outer surface of the wall may be similarly shaped and/or substantially extend in parallel in a longitudinal section of the cavity. For example, the guiding device may be substantially shaped as a cone or comprise a half egg shape. Thereby, accommodation of an outer portion of the stent device may be facilitated at the outer surface. That is, a portion of the stent device not being received within the cavity (in embodiments, extending from the portion of the stent device being received) may be supported or held at the outer surface, in embodiments, without impairing the biasing of the portion received within the cavity. Thereby, accommodation of the portion to be received in the cavity is facilitated, as it may be ensured that portions not to be initially collapsed are arranged and/or held outside of the cavity, in embodiments in a substantially unbiased manner.

The diameter of the opening and the inner diameter of the through-hole are typically equal. As such, substantially no gap is foreseen between the diameter of the opening and the directly adjacent inner diameter of the through-hole of the first cylindrical portion. Thereby, direct insertion into the through-hole may be facilitated. In addition, it may be avoided that the collapsed portion expands within the through-hole of the first cylindrical portion.

The longitudinal extension of the first cylindrical portion may be configured to only accommodate a first inner portion of the stent device. For example, the stent device may comprise an inner stent or body, in embodiments comprising a prosthetic heart valve, and an outer (support or anchoring) frame, wherein the outer frame extends from a longitudinal end portion not being received in the cavity, opening, and through-hole of the first cylindrical portion (e.g. the support or anchoring frame consists in at least three supporting or anchoring arms extending from the inner mesh-shaped body).

The first cylindrical portion may hence be configured for establishing a first collapsing step of a two-step collapsing of the stent device, wherein an outer portion or outer frame may be collapsed in a second step. Typically, the second step does not involve the first cylindrical portion. In embodiments, the outer portion of the stent device may be collapsed around the collapsed first inner portion in a concentric manner.

The longitudinal extension of the first cylindrical portion or through-hole thereof may furthermore be dimensioned such that a predefined length and/or a predefined portion of the first inner portion of the stent device extends beyond the first cylindrical portion in the longitudinal direction and beyond the longitudinal end opposing the opening of the cavity. Such extension may be desirable to provide a coupling with a locking mechanism of a delivery system, e.g. a mandrel. The delivery system may e.g. be configured to engage a particular mesh structure of the stent device.

In embodiments, the guiding device and the first biasing device are configured to bias the stent device, comprising a prosthetic heart valve, into an intermediate collapsed state. As described above, it may hence be foreseen that the collapsing of the stent device is performed by two successive steps. Hereby, the guiding device and the first biasing device are involved in the first step, e.g. in collapsing a first inner portion of the stent device. Such an inner portion may also comprise radially outward projecting features. For example, the guiding device and the first biasing device may facilitate the collapsing of a stent device comprising a prosthetic tricuspid or mitral valve. Accordingly, an inner portion, such as an inner stent device comprising a prosthetic heart valve, may be collapsed in a first step and an outer frame of the stent device may be collapsed in a second step.

In embodiments, the second collapsing step is performed independent from or in the absence of the guiding device and the first biasing device (see below). Thus, the guiding device and the first biasing device may be removed and/or may be decoupled from the stent device in the intermediate collapsed state. An inner sheath may be gradually advanced over the collapsed first inner portion of the stent device via a longitudinal end portion of the first cylindrical portion opposing the opening. Thereby, the intermediate collapsed state is maintained, which typically implies the inner portion or inner stent to be held in a radially inward biased state. In other words, the first biasing device may be decoupled and the guiding device may be longitudinally displaced in a direction away from the inner sheath. That effect may be achieved in such manner that the advancing inner sheath successively covers adjacent portions of the collapsed first inner portion, which are no longer being accommodated by the first cylindrical portion. To maintain a relative position upon deployment, a guide wire of a catheter may be inserted through the stent device. Insertion may occur at least via the first cylindrical portion and opening of the cavity prior to the biasing into the intermediate collapsed state.

The intermediate collapsed state may hence be considered as a “first collapsed state”. The collapsing of the remaining portion of the stent device (e.g. outer portion comprising outer frame, such as support or anchoring frame) provides a final or predefined collapsed state.

The guiding device may comprise a retention surface configured to engage a surface of the first biasing device and to maintain a position of the guiding device relative to a portion of the first biasing device upon actuation of the biasing mechanism. Thereby, a predefined arrangement of the guiding device and the first biasing device in the coupled state with the stent device is maintained. The retention surface may hence secure the guiding device and/or avoid a relative movement of both components in a direction of a force exerted by the biasing mechanism. Typically, the direction of force is the longitudinal direction after an initial coupling or locking or securing of the stent device.

In embodiments, the retention surface is arranged at the level of the opening and/or the first cylindrical portion. In embodiments, the retention surface is formed as a radially extending flange portion. Its provision at the level of the opening and/or the first cylindrical portion, e.g. at the corresponding junction, may ensure improved stability upon actuation of the biasing mechanism. Pivoting of the guiding device relative to the first biasing device may be prevented. The first cylindrical portion may be received and/or accommodated, e.g. surrounded, by the first biasing device or one or more components thereof. Thereby, the retention surface may function as a stop preventing further longitudinal displacement. Alternatively or additionally, the retention surface may function to maintain or to bias the guiding device and the first biasing device in a predefined relative longitudinal arrangement.

A flange configuration may e.g. be established in the form of a disc extending from the outer surface of the guiding device and/or first cylindrical portion. In embodiments, that function is realized at the level of the opening, which typically constitutes a minimal inner diameter of the cavity. For example, a disc-like flange may radially extend from a substantially cone-shaped guiding device. The provision of a flange not only ensures that the guiding device may be secured to the first biasing device. It may also facilitate removal of the guiding device, once a predefined portion of the stent device has been accommodated in the through-hole of the first cylindrical portion. The flange may for example form a gripping portion, which does not directly affect the collapsed portion of the stent device.

The flange-like retention surface may also define a circumferential groove or slot at a radially outer(most) wall portion, which may be formed so as to be surrounded by a wall portion of the surface of the first biasing device being engaged. The surface of the first biasing device being brought into contact with the first guiding device may hence form a recess at least in part accommodating the retention surface. The groove may receive one or more releasable pins or screws extending through a respective hole of the wall of the first biasing device and protruding into the groove, so as to secure the guiding device with respect to the first biasing device, in embodiments, in a form-fitting and/or press-fitting manner.

For facilitating the portion of the stent device to be radially inward biased, the first biasing device may be configured to provide a longitudinal biasing force to the stent device. By the longitudinal biasing force, in embodiments, a tensional force with regard to the stent device, the portion of the stent device to be received by the cavity may be longitudinally displaced and advanced into the through-hole of the first cylindrical portion. The radial biasing may be supported or effected by the shape of the cavity. Hereby a transition from a larger diameter to a smaller diameter of the stent device is ensured. In embodiments, the biasing mechanism is configured to only provide a longitudinal biasing force to the stent device.

It may be desired to advance the stent device into the through-hole of the first cylindrical portion in a longitudinal direction. In this regard, the first biasing device may comprise a slidable component comprising the coupling mechanism, a rotatable component engageable with the slidable component and a sleeve component.

In embodiments, an ensemble of different components as parts of the first biasing device furthermore allows them to be decoupled from each other. Decoupling may occur when the coupling mechanism to the stent device is released. Thereby, the first biasing device is disassembled. A successive biasing and collapsing step, may be facilitated in this manner.

The slidable component is only longitudinally displaced upon rotation of the rotatable component. In other words, the rotatable component may mechanically engage the slidable component in a longitudinal direction, e.g. a proximal direction, but, in embodiments, not in a circumferential direction. As a result, a rotational movement of the rotatable component is translated into a longitudinal movement of the slidable component. The rotation thereby facilitates the biasing in providing an extent of leverage, while an improved control of the longitudinal displacement is simultaneously ensured.

In embodiments, the rotatable component is initially arranged at a distal longitudinal end of the first biasing device. It may be advanced in a proximal direction by rotation, e.g. in a helical manner. The terms “distal” and “proximal” in this regard are to be understood in the context of their common use when applying catheters, The longitudinal end being closest to the patient, e.g. a nose cone on a guide wire, is considered as a “distal end” and a longitudinal end being closest to a surgeon, e.g. a steering knob or handle, is considered as a “proximal end”.

In embodiments, the first biasing device further comprises a sleeve component. The slidable component is slidably arranged within the sleeve component. The rotatable component is arranged at least partially around an outer surface of the sleeve component. The sleeve component may hence provide a coupling or relative arrangement between the slidable component and the rotatable component. For example, the sleeve component may comprise an outer thread to direct the rotatable component during rotation, e.g. in a helical manner. The sleeve component may also facilitate longitudinal displacement of the slidable component into and out of the interior of the sleeve component.

Accordingly, the biasing mechanism may be realized by the rotatable component, the slidable component, and the sleeve component. The coupling mechanism on the slidable component desirably ensures that a longitudinal displacement of the slidable component is translated to the stent device when this one is coupled to said slidable component.

In embodiments, the sleeve component comprises at least one longitudinal slit. The slidable component and the rotatable component are mechanically couplable via the at least one longitudinal slit. The slit or slits hence facilitate that a rotational movement of the rotatable component is translated in a longitudinal displacement (in embodiments, only). For mechanical engagement, the slidable component may comprise at least one radially extending protrusion. Each longitudinal slit may be dimensioned to accommodate a respective protrusion. The rotatable component may be configured to engage the at least one protrusion at an outer surface of the sleeve component. The slidable component may e.g. comprise one or more wedge elements, pins, rods, or blocks, that extend through the respective slit and engage a surface of the rotatable component without impairing rotation thereof.

For example, when mounting the first biasing device, the rotatable component may be positioned in a distal end position while the slidable component has been fully inserted into the sleeve component. In said arrangement, a respective e.g. wedge-like element may be inserted through a respective hole of the rotatable component and via a respective slit so as to be coupled to the slidable component. Each wedge-like element may then be secured to the slidable component, e.g. by a screwing fixation.

Upon rotation of the rotatable component, the rotatable component hence engages the one or more protrusions or wedge-like elements received in the respective slit(s), thereby exerting (only) a longitudinal force on the respective protrusion within the respective slit. The longitudinal force may e.g. result from screwing or helical movement towards a proximal end, causing a longitudinal displacement of the slidable component relative to the sleeve component. Accordingly, the rotatable component may press one or more wedge-like elements or blocks received in the respective slit downward while performing a screwing movement. Such engagement may e.g. be provided by a rim-like portion of the rotatable component adapted to be in physical communication with the respective protrusion while allowing rotation along an outer thread of the sleeve component.

It may be envisaged to further improve stability of the slidable component within the sleeve component during longitudinal displacement. In embodiments, two or more slits are provided, which are equally spaced apart along the circumference of the sleeve component. The slit or slits may be considered as groove-like cut-outs in a circular or ellipsoid wall. They may accordingly form one or more longitudinal legs of the sleeve component. They may protrude from a distal end portion of the sleeve component. Analogously, the one or more protrusions are also arranged at a distal end portion of the slidable component, so as to reduce the required longitudinal dimensioning of the first biasing device.

One or more slits and protrusions may furthermore facilitate a decoupling or disassembly of the first biasing device. That effect may be e.g. desired, once an intermediate collapsed state of the stent device has been achieved. For example, the slidable component may be decoupled from the sleeve by longitudinal displacement towards the respective proximal end. Finally, each of the one or more protrusions may be disconnected or does no longer engage the respective slit.

In embodiments, to secure the first biasing device to the guiding device, the sleeve component comprises a support surface. The support surface is configured for providing a mechanical coupling with the guiding device in the assembled state. In embodiments, the support surface is arranged at a distal end portion of the sleeve component being closest to the guiding device. Hereby, significant leverage is avoided while the required dimensions of the loading system are reduced. In embodiments, the support surface is configured to engage a retention surface of the guiding device. The coupling of the support surface with the guiding device or retention surface thereof may allow no relative movement between the guiding device and the sleeve component to occur upon actuation of the biasing mechanism and in the assembled state of the loading system.

The mechanical coupling between the guiding device and the sleeve component may be stabilized. In this regard the support surface may comprise at least one curvature in a longitudinal section configured to receive and accommodate the retention surface. In embodiments, the support surface may be formed as a substantially radially extending flange, e.g. extending from an outer wall portion of the sleeve component.

The at least one curvature of the support surface of the sleeve component may be configured such that its geometry matches with the retention surface, e.g. a similarly shaped outer wall portion of the guiding device. In particular, the mechanical coupling may be provided in a form-fitting manner in the assembled state and the coupled state of the stent device. For example, such form-fitting may be realized by a matching of the geometry between the retention surface and the supporting surface in the assembled state of the loading system and the coupled state and/or pre-biased state of the stent device. The support surface may e.g. have a generally concave shape while the retention surface may comprise a matching convex shape or vice versa. In embodiments, the at least one curvature may be configured such that a concentric arrangement is established at least by the first cylindrical portion and the slidable component. A biasing force exerted by the biasing mechanism may thus be directly translated in the longitudinal direction. As described above, the retention surface of the guiding device may also be accommodated within a corresponding recess of the biasing device, e.g. a recess defined by the supporting surface.

The coupling between the stent device and the coupling mechanism of the slidable component may be facilitated. The slidable component may be formed as a hollow cylinder defining a longitudinal through-hole. In other words, the slidable component may have a substantially tube-like structure. Therein, opposing ends comprise an opening, such that e.g. a guide wire of a catheter may be inserted through the slidable component and the first cylindrical portion towards the opening and into the cavity for being coupled to the stent device. The slidable component may have an inner diameter that is larger than an outer diameter of the first cylindrical portion. in embodiments, the slidable component may surround at least a portion of the first cylindrical portion.

Similarly, the sleeve component may be configured for receiving and accommodating the first cylindrical portion of the guiding device and/or for biasing at least the first cylindrical portion and the slidable component in a concentric arrangement in the assembled state. The sleeve component, slidable component, and/or the first cylindrical portion may hence be structured as a concentric arrangement. The first cylindrical portion is surrounded by the slidable component and the slidable component is surrounded by the sleeve component, at least in the assembled and coupled state and upon activation of the biasing mechanism.

To facilitate the coupling with the stent device, the coupling mechanism may comprise at least one retention groove, cut-out, and/or recess configured to secure a coupling element of the stent device in a form-fitting manner at least in the longitudinal direction away from the cavity. A coupling element of the stent device, e.g. a wire, suture, or flexible and/or releasable connector, may e.g. be guided and extend through the first cylindrical portion and a slidable component so as to be coupled to the coupling mechanism, e.g. by a retention feature forming a stop. For example, a proximal end of the coupling element may comprise a retention feature which is inserted into and engages a cut-out or groove at a proximal end of the slidable component. An initial retention may be foreseen e.g. by a resilient force of the coupling element and or an initial biasing of the biasing mechanism. Upon further actuation of the biasing mechanism, the retention feature, e.g. a block element, may be held and secured into e.g. the groove, e.g. by a rim portion thereof.

Decoupling may e.g. be facilitated by a pulling force onto the coupling element and retention feature in a longitudinal direction opposing an advancing direction of the stent device. This approach may enable disassembly of the slidable component, the sleeve component, and/or the rotatable component in a biased state of the stent device or portion thereof. In embodiments, at least two retention features and/or coupling elements are provided, which may be equally spaced apart along a circumference, e.g. of the slidable component. Thereby, a more equal distribution of the biasing force to be applied to the stent device may be established. The risk of misalignments upon actuation of the biasing mechanism is thus reduced.

In embodiments, the loading system comprises a second biasing device configured for receiving an opposing longitudinal end of the stent device and/or for receiving a portion of the stent device in an intermediate collapsed state. In particular, the second biasing device may be configured for engaging e.g. an outer frame as a second (outer) portion of the stent device. The second biasing device may e.g. be configured for biasing an outer frame of a stent device that is connected to an inner frame or inner stent at the “opposing longitudinal end”. The “opposing longitudinal end” is to be understood as a longitudinal end of the stent device not being received by the cavity of the guiding device, at least not upon initiating the biasing mechanism of the first biasing device. The second (outer) portion of the stent device for example is an outer (support or anchoring) frame.

The second biasing device may hence be configured for biasing a portion, e.g. an outer frame, being in an expanded state around a portion of the stent device being in a collapsed state. The portion being in the collapsed state may be a first inner portion or inner stent of the stent device, which has been radially biased by the biasing mechanism of the first biasing device and has been accommodated by the first cylindrical portion. According to an embodiment, said first inner portion or inner stent is comprising the prosthetic heart valve. In embodiments, the loading system is configured, such that after biasing the first inner portion, the first biasing device and/or guiding device may be gradually removed. Meanwhile, an inner sheath is advanced over the directly adjacent collapsed inner portion, resulting in an intermediate collapsed state. The first inner portion is accommodated within the inner sheath and a second outer portion is present in an expanded state from the opposing longitudinal end.

To bias the outer portion, e.g. an outer frame, of the stent device engaging the second biasing device, the second biasing device may comprise a second cylindrical portion defining a through-hole. The through-hole may have a predefined inner diameter. It is configured for radially biasing an outer portion of the stent device and for accommodating the outer portion of the stent device within the through-hole upon longitudinal displacement of the second cylindrical portion over and relative to the outer portion of the stent device.

Accordingly, the second cylindrical portion may be advanced over e.g. an outer frame portion of the stent device in a proximal direction. Hereby, the outer frame portion is gradually biased radially inward and accommodated within the second cylindrical portion. The relative displacement may furthermore involve a corresponding displacement of the second cylindrical portion relative to a first inner portion of the stent device, which has previously been collapsed and is e.g. accommodated within an inner sheath. The second outer portion and the first inner portion may e.g. be successively biased in a concentric manner, thereby reducing the overall dimensions of the collapsed stent device, more particularly, the stent device comprising a prosthetic heart valve. Insertion and deployment of the stent device into the patient is thus facilitated.

Accordingly, the inner diameter of the second cylindrical portion may be larger than or may substantially correspond to an outer diameter of the first cylindrical portion. The inner diameter of the second cylindrical portion may e.g. be adapted so as to accommodate for a predefined radial extension, in the collapsed state, of an outer frame portion of the stent device and/or the radial extension of an inner sheath extending (slightly) beyond the outer diameter of the first cylindrical portion.

The biasing exerted by the second biasing device may be facilitated. The second biasing device may comprise a ring-shaped securing device being releasably couplable to the second cylindrical portion. The ring-shaped securing device may comprise first and second ring-shaped elements. The ring-shaped securing device, and more specifically the first ring-shaped element, may also be configured for engaging a stop formed by the second cylindrical portion only in one longitudinal direction. The stop may be formed e.g. as a radially extending flare or rim portion. In embodiments, at a proximal end of the second cylindrical portion.

Upon movement of the ring-shaped securing device towards the outer portion of the stent device to be biased, typically in a proximal direction, an interlocking between the ring-shaped securing device and the second cylindrical portion is established via said stop. Thereby, longitudinal movement of the second cylindrical portion is facilitated. In embodiments, a longitudinal translation of the ring-shaped securing device hence results in a corresponding longitudinal translation of the second cylindrical portion over the portion of the stent device to be biased. For ensuring interlocking and/or engagement with the stop, the ring-shaped securing device may comprise e.g. an inner flange or cover portion, which typically extends over the stop and is in contact with the stop in the coupled state. In embodiments, an open portion of the ring-shaped securing device, e.g. an inner channel or through-hole may be configured for receiving the second cylindrical portion in a slidable arrangement. Hereby, insertion of the second cylindrical portion into the ring-shaped securing device is enabled, until the stop engages the ring-shaped securing device.

In embodiments, the engagement with the stop is reversible. Movement in the opposing longitudinal direction results in decoupling of the ring-shaped securing element and the second cylindrical portion. Disassembly of the second biasing device may hence be provided by simply changing of direction of movement. For example, the stop may engage the ring-shaped securing device in a proximal to distal direction. Decoupling may be provided by corresponding longitudinal displacement relative to the ring-shaped securing device in the distal to proximal direction.

The ring-shaped securing device hence assists the radially inward biasing of the stent device. Thereby, longitudinal advancing of the second cylindrical portion is enabled, e.g. over a corresponding outer portion of the stent device.

The ring-shaped securing device, and more particularly the second ring-shaped element, may furthermore comprise two or more grooves at a longitudinal end face engaging the stent device. The grooves are spaced-apart from each other in a circumferential direction. Each groove is configured for securing a respective supporting or anchoring arm of the stent device in a circumferential direction. In embodiments, the grooves extend in a longitudinal and radial direction. Thereby, the grooves may be adapted to the dimensioning and shape of the respective support/anchor arm.

For example, the supporting or anchoring arms may be part of an outer frame portion of the stent device, which may be adapted to provide anchoring means in the deployed state. Such supporting or anchoring arms may be formed as respective petals extending from the corresponding longitudinal end. They may, e.g. form a ventricular and/or atrial support and may secure an in situ position of an inner portion of the stent device comprising a prosthetic heart valve, e.g. an inner stent. While applying the second biasing device, the grooves may support preventing a twisting and/or intertangling of the support/anchor arms. Thus, a relative position is maintained to facilitate the longitudinal displacement of the second cylindrical portion.

In embodiments, the grooves are formed to engage and bias the respective support/anchor arm in a longitudinal and/or radial direction upon longitudinal displacement of the ring-shaped securing device relative to the portion of the stent device to be biased. The grooves may hence facilitate the accommodation of the (second) portion of the stent device into the second cylindrical portion, e.g. by enabling a pre-biasing and/or radial compression of the corresponding portion of the stent device.

Each groove may furthermore comprise a respective first pin configured for securing the respective support/anchor arm of the stent device in the circumferential direction. The first pins may facilitate the securing of the respective support/anchor arm even in case e.g. the support/anchor arms have varying circumferential extensions along their longitudinal extension and/or at different collapsed states. For example, the support/anchor arms may have a tapered shape and/or gradually downsizing circumferential extensions. It may occur that contact with the groove surface is reduced at a collapsed state preceding a final or predefined collapsed state and an intermediate collapsed state, e.g. in case from 70% to 95% of the longitudinal extension of an outer portion of the stent device has been collapsed. In such instance, the first pins may ensure that contact with the stent device portion not yet being collapsed is maintained so as to secure the relative positions of the supporting or anchoring arms.

The ring-shaped securing device may adopt a variety of configurations. In embodiments, the ring-shaped securing device is formed by a first ring-shaped element and a second ring-shaped element that are releasably couplable. Hence, the ring-shaped securing device may be formed as a two-part device which may be assembled and disassembled. For example, the first ring-shaped element may be configured for providing an enlarged surface and/or cross-sectional area. It may facilitate the longitudinal displacement of the ring-shaped securing device, while the second ring-shaped element may e.g. be configured for enabling a mechanical coupling with the second cylindrical portion, e.g. by engaging a stop of the second cylindrical portion. Such an engagement may be ensured e.g. by a reduced inner diameter or radially inward extending flare compared with a radially inward extension of the second ring-shaped element. The second ring-shaped portion may also comprise two or more grooves configured for receiving a respective support/anchor arm of the stent device, as described above.

Functionalities of the ring-shaped elements and the decouplable configuration allow functions of the ring-shaped device to be optimized and adapted to the shape and dimensions of the stent device. Such functions may e.g. facilitate longitudinal displacement of the second cylindrical portion and/or secure support/anchor arms of a portion of the stent device. A two-part configuration ensures that the ring-shaped securing device may be easily decoupled. Such a configuration may be particularly desirable to remove the second biasing device, once the stent device has been collapsed into the predefined state and e.g. an outer sheath is to be advanced over an outer collapsed portion of the stent device.

In embodiments, the first and second ring-shaped elements are decouplable by a relative displacement in the longitudinal direction. In other words, the ring-shaped elements may be simply decoupled by movement of the respective ring-shaped elements into opposing longitudinal directions. For example, the first ring-shaped element may be configured such that a predefined movement in a distal direction relative to the second ring-shaped element and/or a predefined movement of the second ring-shaped element in a proximal direction may result in a decoupling of the ring-shaped securing device.

For example, the second ring-shaped element may at least partially be accommodated by a recess of the first ring-shaped element in the coupled state. The first ring-shaped element may e.g. define a stop, e.g. an inner circumferential extension. Hereby, a longitudinal displacement of the second ring-shaped element beyond the first ring-shaped element is avoided. Accordingly, the first and second ring-shaped elements are configured such that a decoupling is only enabled when moving the ring-shaped elements in predefined particular directions. Meanwhile, said ring-shaped elements are maintained in a coupled state when moving at least one of the ring-shaped elements in the opposing direction. Thereby, a simple decoupling mechanism may be realized while ensuring that longitudinal displacement of the second cylindrical portion is enabled, when moving the ring-shaped elements in the corresponding direction.

Decoupling of the ring-shaped securing device may be further facilitated. In embodiments, the second ring-shaped element is formed of equally formed parts that are releasably couplable to each other. Accordingly, once the first and second ring-shaped elements have been decoupled, the second ring-shaped element may be easily disassembled. As a result, the ring-shaped securing device may be more easily removed. The second ring-shaped element may hence be formed of multiple components that may also be releasably interlocked and mounted. Thereby, assembly of the ring-shaped securing device and relative movements between the second ring-shaped element and first ring-shaped element may be facilitated.

Coupling between the first and second ring-shaped elements may be established by mechanical features providing an interlocking of said ring-shaped elements. In embodiments, the second ring-shaped element may comprise second pins dimensioned and arranged to extend through and beyond holes in the first ring-shaped element in the coupled state of the first and second ring-shaped elements. In particular, the second pins may be circumferentially spaced apart and configured to rotationally secure the first and second ring-shaped elements relative to each other in said coupled state.

For example, the second pins or other protrusions may have a shape corresponding to the respective holes. Hereby, a form-fitting or interference fit between the first and second ring-shaped elements is established. In embodiments, the second pins are formed as substantially cylindrical or rectangular pins. However, other polygonal or ellipsoid shapes may also be provided. In embodiments, the circumferential spacing is equal for adjacent pins so as to provide a rotational symmetry and facilitate the assembly or coupling of the first and second ring-shaped elements.

Second pins extending beyond the holes of the first ring-shaped element may exhibit another aspect. Second pins may be accessed after assembly. Thereby, second pins may be used to at least partially separate the second ring-shaped element from the first ring-shaped element, i.e. by pressing of the second pins in the corresponding longitudinal direction, e.g. in the proximal direction, in the assembled state. Accordingly, the second ring-shaped element may be moved in a proximal direction relative to the first ring-shaped element by second pins.

A mechanical coupling may also be foreseen, alternatively or in addition, by a configuration of the second ring-shaped element comprising holes dimensioned and arranged for receiving and accommodating respective first pins of the first ring-shaped element. As described above, first pins may be adapted to at least partially secure respective support/anchor arms of the stent device. In embodiments, first pins hence may extend through the holes of the second ring-shaped element. In particular, such holes may be introduced into respective grooves configured for receiving a respective support/anchor arm of the stent device, as described above.

Depending on the collapsed state, it may, however, be desirable to release such securing or engaging functionality of the first pins. For example, it may be envisaged to avoid such securing of the support/anchor arms at an end stage of the collapsing, e.g. to advance the second cylindrical portion over the remaining portion of an outer portion of the stent device. For such a scenario, a relative movement of the first and second ring-shaped elements in opposing longitudinal directions may establish that the first pins no longer extend through the holes. The remaining portion of the stent device, e.g. the remaining portion of the respective support/anchor arms, is either secured by corresponding grooves or is no longer secured.

Such a shifting of the first and second ring-shaped elements may hence enable that the second cylindrical portion may be advanced in the respective longitudinal direction. Hereby, a fully or predefined collapsed state is established. The portion collapsed by the second biasing device may be substantially aligned with the inner diameter of the second cylindrical portion. A gradual advancing of an outer sheath over an end face or end tip portion of the stent device at the proximal end of the second cylindrical portion may be realized.

Once the stent device has been fully collapsed, the second biasing device may be removed. Any further components required for insertion and/or deployment in a patient's body may be coupled or attached to the stent device hence being in the predefined collapsed state.

The loading system of the present disclosure is particularly desirable when the a stent device comprises (a) a mesh-shaped body extending in an axial direction, wherein the body defines an inner portion (in embodiments, providing a passageway from a proximal end to a distal end of the body) and (b) outer supporting or anchoring frames (also called supporting or anchoring arms), which extend radially outward from the body, i.e. from the distal end of the mesh-shaped body towards the proximal end, wherein each supporting or anchoring frame extends radially outwards. Thus, supporting or anchoring frames may be formed as respective projecting features such as petals extending from the mesh-shaped body. In this case, the loading system of the present disclosure is particularly desirable, as it enables a two steps collapsing process resulting in a predefined collapsed stent device comprising: (a) a first inner portion of the stent device being collapsed in an inner sheath and (b) a second outer portion (or outer frame e.g. support or anchoring frame) of the stent device being collapsed in an outer sheath around the inner sheath. This is particularly desirable during delivery of the stent device in a patient to be treated at a targeted site, for example to replace a heart valve (e.g. aortic, mitral or tricuspid heart valve) as this allows to first deploy the outer portion of the stent device (e.g. support or anchoring frame), to adjust its positioning within the targeted site (e.g. native mitral or tricuspid valve annulus) and finally to deploy the inner portion of the stent device (e.g. containing a prosthetic valve, e.g. a prosthetic tricuspid valve) within the targeted site.

The different elements of the loading system can be made of any material known by the skilled person in this art, they can be made of polymer such as polypropylene (PP), Polyphenylsulfone (PPSU), Polyether ether ketone (PEEK), 3D printing material such as Acrylonitrile Butadiene Styrene (ABS) or Polytetrafluoroethylene (PTFE).

The present disclosure further relates to a method for collapsing stent device (i.e. in embodiments, crimping the stent device from an expanded diameter to a compressed one), wherein the stent device comprises an inner and an outer portion and further comprises a prosthetic heart valve, the method comprising the steps of:
assembling the components of the first biasing device with the guiding device;
connecting the stent device to the first biasing device by way of a coupling mechanism;
pulling the stent device through the cavity into the first cylindrical portion of the guiding device wherein the inner portion of the stent device is radially inward biased leading to intermediate collapsed state of the stent device;
advancing an inner sheath over the collapsed inner portion of the stent device;
releasing the guiding device and the first biasing device;
placing and pushing the second biasing device over the outer portion of the stent device, wherein the outer portion of the stent device is radially inward biased leading to predefined collapsed state of the stent device; and
advancing an outer sheath over the collapsed outer portion of the stent device.

### BRIEF DESCRIPTION OF THE DRAWINGS

The present disclosure will be more readily appreciated in further detail by reference to the following detailed description when being considered in connection with the accompanying drawings in which:

FIG. 1 is a schematic side view of a guiding device of the loading system according to the present disclosure;

FIG. 2 is a perspective top view of the guiding device according to FIG. 1 in a coupled state with a first biasing device;

FIG. 3 is a schematic side view of the loading system according to the present disclosure during assembly;

FIG. 4 is a schematic side view of the loading system according FIG. 3 in the coupled and assembled state with a stent device comprising a prosthetic heart valve;

FIG. 5 is a schematic side view of the first biasing device of the loading system according to the present disclosure with a longitudinal translation;

FIG. 6 is a schematic side view of a coupling mechanism of the loading system according to the present disclosure;

FIG. 7 is a schematic side view of the first biasing system in an intermediate collapsed state of the stent device comprising a prosthetic heart valve;

FIG. 8 is a schematic perspective side view of a second biasing device of the loading system according to the present disclosure;

FIG. 9 depicts the second biasing device according to FIG. 8 in a side view;

FIG. 10 is a schematic perspective side view of a first ring-shaped element of a ring-shaped securing device of the second biasing device according to FIG. 8;

FIG. 11 is a schematic side view of a second cylindrical portion of the second biasing device according to FIG. 8;

FIG. 12A depicts a second ring-shaped element of a ring-shaped securing device of the second biasing device according to FIG. 8;

FIG. 12B depicts the second ring-shaped element of a ring-shaped securing device of the second biasing device according to FIG. 8;

FIG. 13 shows the top perspective schematic view of the second biasing device according to FIG. 8 engaging an outer portion of the stent device comprising a prosthetic heart valve;

FIG. 14 shows the second biasing device according to FIG. 13 in a schematic side view in a further collapsed state of the stent device comprising a prosthetic heart valve;

FIG. 15 shows the second biasing device according to FIG. 14 in a schematic perspective side view in a further collapsed state of the stent device comprising a prosthetic heart valve;

FIG. 16 shows the second cylindrical portion of the second biasing device according to FIG. 15 in a further collapsed state of the stent device comprising a prosthetic heart valve;

FIG. 17 shows an alternative embodiment of the guiding device depicted in FIG. 1 with a corresponding sleeve component;

FIG. 18 shows details of rotatable component and protrusions of a slidable component of the first biasing device;

FIG. 19A shows details of a predefined collapsed state of the stent device with an outer sheath;

FIG. 19B shows details of the predefined collapsed state of the stent device with an outer sheath; and

FIG. 20 shows an exemplary collapsible stent device compatible with the loading system according to the present disclosure.

FIG. 21 shows details of the second biasing device.

### DETAILED DESCRIPTION

In the following, the present disclosure will be explained in more detail with reference to the accompanying figures. In the Figures, corresponding elements are denoted by identical reference numerals and repeated description thereof may be omitted in order to avoid redundancies.

In FIG. 1 an embodiment of a guiding device 10 of the loading system according to the present disclosure is schematically depicted in a side view. The guiding device 10 comprises a first cylindrical portion 12, which comprises an inner through-channel extending in a longitudinal direction. The first cylindrical portion 12 extends from a conical portion defining an equally shaped inner cavity (not shown) that is open and configured to receive and accommodate a portion of a stent device to be biased into a collapsed state. The inner cavity and the through-hole of the first cylindrical portion 12 are connected via an opening (not shown) at a junction between the inner cavity and the first cylindrical portion 12, i.e. at the level of the cavity having the smallest inner diameter.

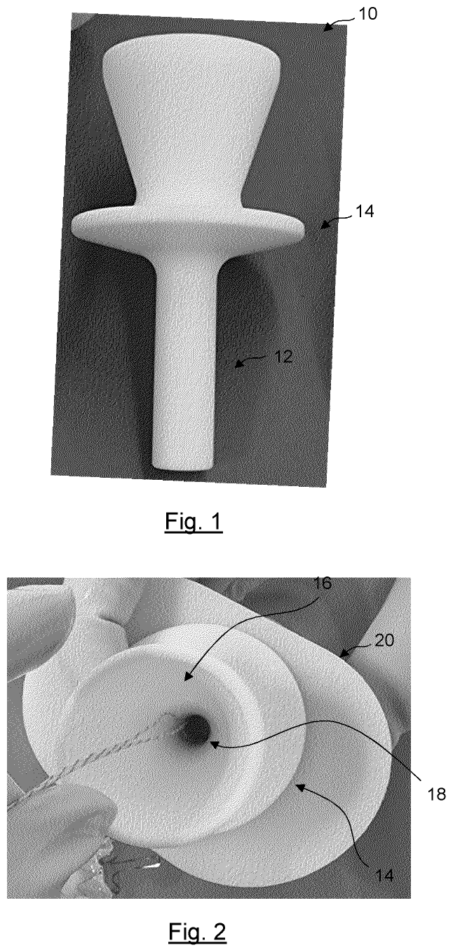

The open cavity and the through-hole of the first cylindrical portion 12 are hence in fluid communication. This enables that the stent device portion being received in the cavity may be advanced towards the through-hole and be accommodated therein. As further explained below in view of FIG. 2, the shape of the cavity and the conical portion provides a continuous inner surface defining a guiding surface with a gradually reducing diameter from a free longitudinal end portion towards the opposing longitudinal end portion extending into the first cylindrical portion 12. This allows that the received portion of the stent device may be biased radially inward before being inserted into the through-hole of the first cylindrical portion, such that the accommodation of the portion of the stent device within the through-hole is facilitated and the portion of the stent device is biased in a collapsed state.

Between the conical portion and the first cylindrical portion 12 a retention surface 14 is provided, which is substantially formed as a radially outward extending flange or disc. The retention surface 14 is configured to engage a corresponding surface of a first biasing device 22 of the loading system in an assembled state and is shaped and dimensioned to provide a predefined relative arrangement between the guiding device 10 and the first biasing device 22. As shown, in the present, non-limiting example, the retention surface 14 comprises a curvature and defines an overall funnel shape of the conical portion and the retention surface 14, facilitating proper positioning and coupling of the guiding device 10 during assembly.

In FIG. 2 the open cavity 16 of the conical portion is shown in further detail. From the free longitudinal end of the cavity 16 comprising the largest inner diameter, a stent device may be inserted into the cavity 16 towards an opening 18 defining the smallest inner diameter of the cavity 16. The end portion or end face of the cavity 16 having the largest inner diameter may be considered a distal end while the end of the cavity 16 having the smallest diameter may be considered a proximal end, as seen from the perspective of a distal end of a catheter to be coupled with the stent device.

The gradually reducing diameter of the cavity 16 provides a guiding and support surface for the portion of the stent device being received, such that a longitudinal biasing force being exerted onto the stent device results in a longitudinal displacement of the portion of the stent device towards the opening 18 and into the through-hole of the first cylindrical portion 12.

To facilitate such biasing, the stent device may comprise a coupling element, such as a removable suture having a locking feature, wherein the coupling element may be inserted through the opening 18 and the through-hole of the first cylindrical portion 12 so as to provide a biasing force acting in the longitudinal direction of the guiding device 10 and the first biasing device.

The guiding device 10 is shown in an assembled or coupled state with a first biasing device, wherein, according to the present, non-limiting example, the retention surface 14 is coupled with a corresponding support surface 20 of the first biasing device. The coupling between the retention surface 14 and the support surface 20 not only ensures that a relative position may be secured, but also facilitates handling of the loading system by forming an enlarged gripping portion and/or providing some extent of leverage.

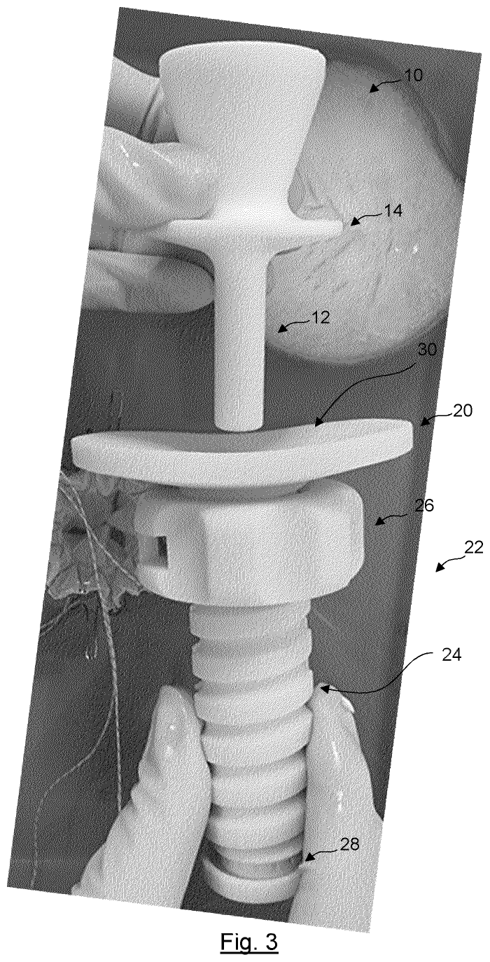

The assembly of the guiding device 10 and the first biasing device 22 is further depicted in FIG. 3 in a schematic side view of the loading system according to the present disclosure. The first biasing device 22 comprises a sleeve component 24 being formed as a cylindrical portion with an inner through-hole and longitudinally extending from the support surface 20. The sleeve component 24 hence forms an open and continuous cavity or channel, which is adapted to receive and accommodate the first cylindrical portion 12 of the guiding device 10 in the coupled state.

Arranged within the sleeve component 24 is a slidable component 28, which may be (only) longitudinally displaced relative to the sleeve component 24. The slidable component 28 comprises a coupling mechanism (not shown) at a respective longitudinal end opposing the support surface 20 in the coupled state of the guiding device 10 and the first biasing device 22. The coupling mechanism is configured to couple the slidable component 28 with the stent device being received in the cavity 16, e.g. by a coupling element having a locking feature as described above. Thereby, upon longitudinal displacement in a proximal direction, a longitudinal biasing force may be exerted onto the stent device, such that a corresponding portion of the stent device may be advanced towards the opening 18 and into the through-hole of the first cylindrical portion 12 while being biased radially inwardly with support of the shape and dimensions of the cavity 16.

The longitudinal displacement of the slidable component 28, in particular in the proximal direction, is facilitated by a rotatable component 26, which is arranged along an outer circumference of the sleeve component 24. Rotation of the rotatable component is facilitated by a helical thread formed on the outer surface of the sleeve component 24, which enables that the rotatable component 26 may be rotated along a longitudinal axis of the first biasing device 22 and the sleeve component 24 thereof and be gradually advanced in the longitudinal, e.g. proximal, direction due to the thread angle.

The rotatable component 26 engages the slidable component 28 via one or more grooves or longitudinal slits (not shown), which are configured to receive a respective protrusion, e.g. a wedge-like element, block, or pin (not shown), extending radially from the outer surface of the slidable component 28. In the assembled state said one or more protrusions or pins extend radially outward through the respective groove(s) or slit(s) so as to be engaged by a corresponding surface of the rotatable component 26. For example, the rotatable component 26 may be formed and dimensioned such that a rim portion may engage the one or more protrusions in a longitudinal direction without engaging said one or more protrusions in a circumferential direction. Thereby, rotation of the rotatable component 26 is not impaired by the one or more protrusions. Accordingly, upon rotation of the rotatable component 26, the rotatable component 26 engages the one or more protrusions, resulting in a longitudinal displacement of the slidable component 28 that corresponds to the helical and corresponding longitudinal displacement of the rotatable component 26. Thereby, a biasing force is provided, which is facilitated by the sleeve component 24, the rotatable component 26, and the slidable component 28, together forming a biasing mechanism.

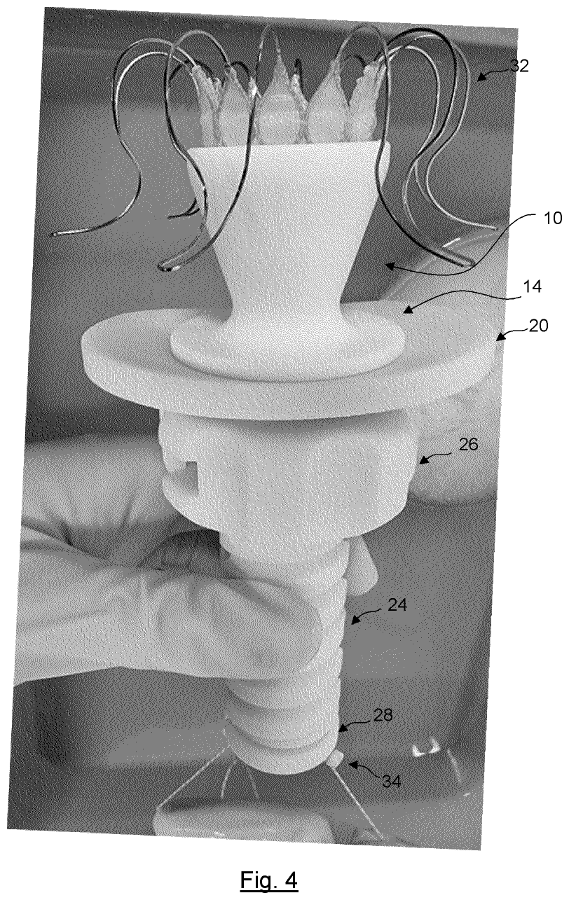

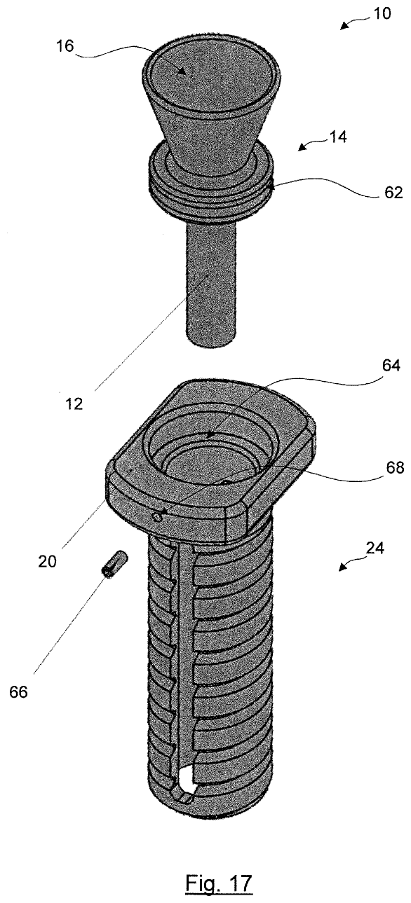

To facilitate the coupling of the guiding device 10 and the first biasing device 22, the support surface 20 may comprise a curvature 30 in the longitudinal section of the first biasing device 22. As will be shown in further detail in FIG. 4, said curvature 30 is configured to receive and accommodate the retention surface 14 of the guiding device 10. The curvature 30 may provide a form-fitting engagement, preventing longitudinal displacement of the guiding device 10 beyond the support surface 20 in a proximal direction and optionally preventing rotation of the guiding device 10 relative to the first biasing device 22 in the assembled state of the guiding device 10 and the first biasing device 22 and the coupled state of the stent device received in the cavity 16. An alternative embodiment of the retention surface 14 of the guiding device 10 and the support surface 20 of the first biasing device 22 is depicted in FIG. 17, as described below.

Such assembled and coupled state is depicted schematically in FIG. 4 in a side view of the loading system. Accordingly, the first cylindrical portion 12 has been inserted into the sleeve component 24, such that the retention surface 14 of the guiding device 10 engages the support surface 20 of the first biasing device 22. The rotatable component 26 has been positioned at the distal end of the sleeve component 24, i.e. adjacent to the support surface 20. The slidable component 28 has accordingly been inserted into the sleeve component 24 up to a rim portion at a longitudinal end of the slidable component 28. Said rim portion defines a coupling mechanism 34 configured to releasably couple and secure a coupling element of the stent device (e.g. a removable suture having a locking feature), as shown in further detail in view of FIG. 6.

In FIG. 4, a portion of a stent device comprising a prosthetic heart valve is received and accommodated in the cavity 16 of the guiding device 10. According to the present example, an inner stent portion or first inner portion is introduced into the cavity 16 while an outer frame portion or second outer portion 32 of the stent device is arranged along an outer conical surface of the guiding device 10. Due to the coupling mechanism 34 and the biasing mechanism, the inner portion of the stent device may be advanced towards the opening 18 and into the through-hole of the first cylindrical portion 12 upon actuation of the biasing mechanism, i.e. rotation of the rotatable component 26 and corresponding longitudinal displacement of the slidable component 28 in the proximal direction, according to the present example. This is because the coupling mechanism 34 ensures that a corresponding longitudinal displacement of the coupling element and hence the inner portion of the stent device is provided while the guiding and support surface of the cavity 16 facilitates or enables a radially inward biasing.

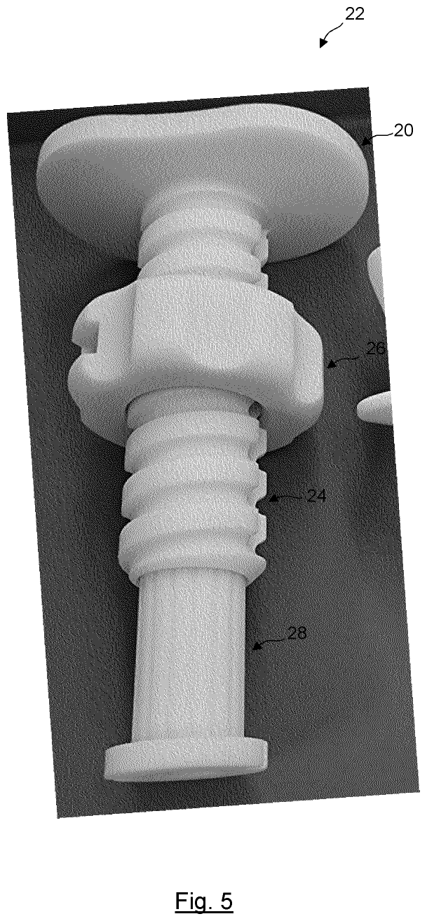

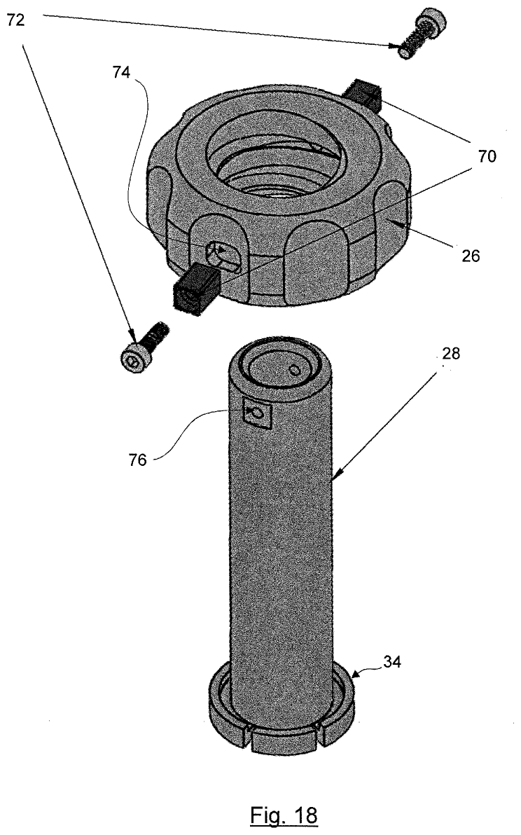

A longitudinal displacement or translation and interaction of the slidable component 28 with the rotatable component 26 is depicted in FIG. 5 showing only the first biasing device 22 of the loading system according to the present disclosure. The first biasing device 22 has not been coupled to the guiding device 10 and no coupling with a stent device is shown. In this side view, however, a longitudinal displacement of the slidable component 28 is clearly visible and results from a corresponding rotational movement and longitudinal displacement of the rotatable component 26 along the helical thread provided on the outer surface of the sleeve component 24. The mechanical engagement of the rotatable component 26 with the slidable component 28 via said sleeve component 24 is provided via a groove or slit, which is shown in and described in further detail in view of FIG. 7. Further details of a mechanical engagement according to an embodiment are furthermore depicted in and described in view of FIG. 18.

The coupling mechanism 34 is depicted in further detail in FIG. 6, showing the sleeve component 24 and the slidable component 28 in a schematic side view. As shown, the coupling mechanism 34 may comprise a rim portion and one or more grooves configured to accommodate a coupling element 36. In the present, non-binding example, the coupling element 36 comprises a connector, e.g. formed by a suture, and an insert or securing or locking feature, which engages the rim portion and groove and is secured within said groove at the rim portion, when applying a longitudinal force in the proximal direction to the slidable component 28. The coupling mechanism 34 and securing of the coupling element 36 may be released upon movement of the coupling element in the distal direction, e.g. facilitated by a resilience of the coupling element 36 or connector thereof, such that the coupling element 36 may be released from the groove and/or rim portion of the coupling mechanism 34.

In FIG. 7 an example is shown, wherein a stent device is in an intermediate collapsed state, e.g. wherein the first inner portion of the stent device has been collapsed and is accommodated within the through-hole of the first cylindrical portion 12. In this Figure, the slits 38 of the sleeve component 24 are shown revealing an end tip or end portion of the inner portion of the stent device, e.g. an atrial flare, which extends out of the through-hole of the first cylindrical portion 12. Said end tip or end portion may be connected to a connecting feature of a catheter, which may be advanced towards the stent device via a guide wire received within the sleeve component 24 and which has been introduced through the stent device prior to actuation of the biasing mechanism.

In the intermediate collapsed state, the rotatable component 26 has been accordingly advanced in the proximal direction via the thread of the sleeve component 24. Once the intermediate collapsed state has been achieved, the coupling mechanism 34 may be released and the first biasing device 22 may be removed by moving the corresponding components in a proximal direction away from the guiding device 10. In the intermediate collapsed state of the stent device the first inner portion of the stent device may be covered by an inner sheath of a delivery system, which may be advanced over said portion of the stent device via a proximal end and by gradually moving the directly adjacent guiding device 10 in a distal direction. Thereby, the first inner portion of the stent device, comprising a prosthetic heart valve, may be provided in a collapsed state within an inner sheath while a second outer portion may be successively brought into a collapsed state by a second biasing device.

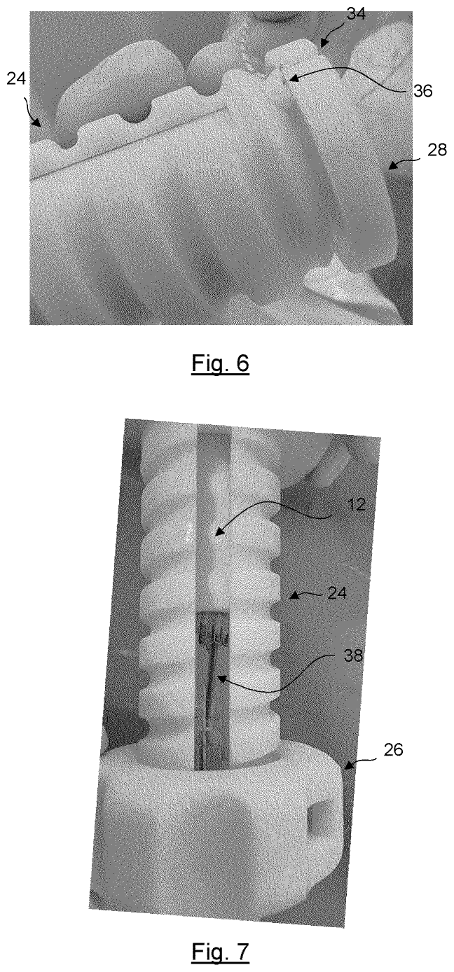

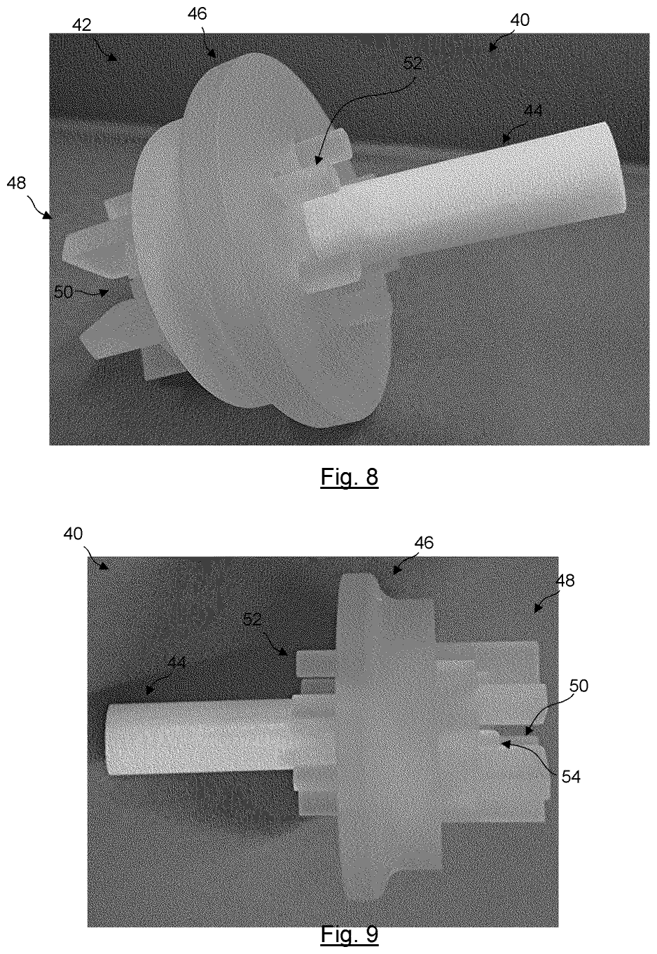

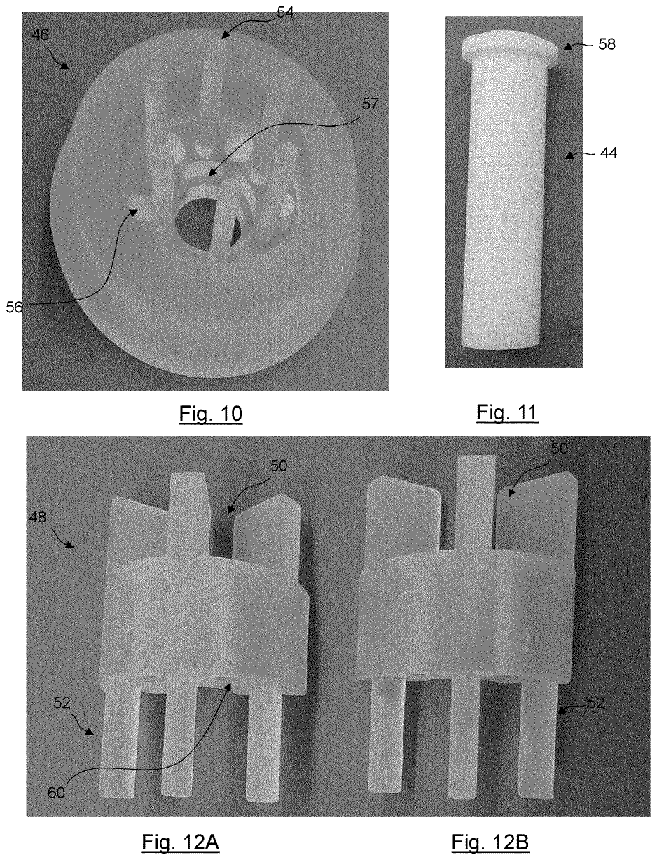

An example of such second biasing device 40 is depicted in FIGS. 8 to 12. Accordingly, the second biasing device 40 comprises a ring-shaped securing device 42 and a second cylindrical portion 44. The ring-shaped securing device 42 is formed of a first ring-shaped element 46 accommodating a second ring-shaped element 48, which may be releasably coupled to each other. The second cylindrical portion 44 extends through a continuous opening or through-hole of the ring-shaped securing device 42 and is releasably couplable with the ring-shaped securing device 42. As best shown in FIGS. 10 and 11, the second cylindrical portion 44 may comprise a stop 58 adapted to engage an inner cover or radially inward flare within a recess 57 of the first ring-shaped element 46. Thereby, the second cylindrical portion 44 remains in the coupled state with the ring-shaped securing device 42 or first ring-shaped element 46 thereof when moving the second biasing device 40 in the corresponding longitudinal direction, e.g. the proximal direction.

Since the second cylindrical portion 44 is slidably arranged within the opening or through-hole, the ring-shaped securing device 42 and first ring-shaped element 46 thereof may be decoupled and removed from the second cylindrical portion 44 when moving the ring-shaped securing device in the opposing longitudinal direction, e.g. the distal direction. Thereby, a coupling and decoupling may be facilitated while at the same time the application of a biasing force towards the portion of the stent device not yet being in a collapsed state may be facilitated by the larger sized ring-shaped securing device 42.

The coupling of the first and second ring-shaped elements 46, 48 is provided by a recess 57 of the first ring-shaped element 46 configured to accommodate a corresponding portion of the second ring-shaped element 48. In addition, the first ring-shaped element 46 comprises a plurality of first pins 54 which are equally spaced apart from each other in a circumferential direction and are configured to be accommodated by and extend through holes 60 of the second ring-shaped element 48. The first pins 54 extend through and beyond the holes 60 and are received within a respective groove 50 formed by the second ring-shaped element 48. By the first pins 54 and the grooves 50 corresponding support arms of an outer portion or outer frame of the stent device may be secured, as described below in view of FIGS. 13 to 15.

The second ring-shaped element 48 also comprises second pins 52 configured to be accommodated by and extend through and beyond corresponding holes 56 in the first ring-shaped element 46. In embodiments, said second pins 52 and holes 56 are also equally spaced apart in the circumferential direction and may be arranged between adjacent first pins 54 and holes 60. The first pins 54 and second pins 52 provide that the first ring-shaped element 46 and the second ring-shaped element may be releasably coupled yet may be rotationally secured relative to each other in the coupled state.

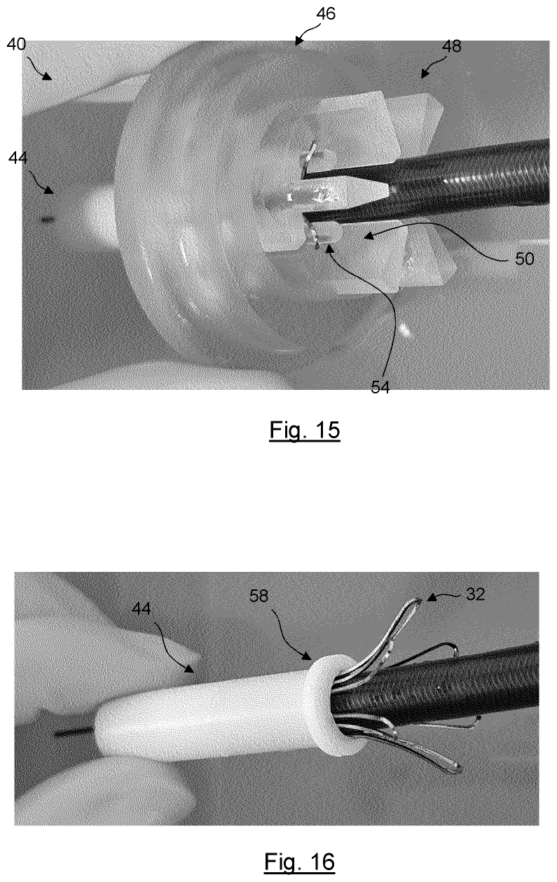

In addition, the second pins 52 may provide that the second ring-shaped element 48 may be longitudinally displaced relative to the first ring-shaped element 46 even while biasing the portion of the stent device to be brought into a collapsed state by the second biasing device 40. Such relative displacement may provide that the first pins 54 may no longer extend through the corresponding holes 60 and/or no longer engage corresponding features of the stent device, as described in further detail in view of FIGS. 15 and 16.

As shown in FIGS. 12A and 12B, the second ring-shaped element 48 may be formed of multiple parts that are couplable to each other and to the first ring-shaped element 46 via the corresponding second pins 52 and first pins 54. In the present example, the second ring-shaped element 48 is formed of two equally formed parts. Together with the first ring-shaped element 46, the parts of the ring-shaped second element 48 according to a configuration form a rotationally symmetric ring-shaped securing device 42.

The second biasing device 40 is shown in an assembled state in FIGS. 13 to 15, wherein the second cylindrical portion 44 and the grooves 50 and/or first pins 54 engage respective portions of an outer portion 32 or outer frame extending from a longitudinal end of an inner portion opposing a longitudinally end being connected to a connector of a catheter. As shown, the inner portion of the stent device has been previously collapsed and an inner sheath of a delivery system has been advanced over the collapsed inner portion. The second biasing device 40 hence engages the remaining outer portion 32 of the stent device in an intermediate collapsed state, wherein the guiding device 10 and the first biasing device 22 have been removed.

Upon engagement of the second biasing device 40 with the outer portion 32 of the stent device, support arms of the outer portion 32 are received by respective grooves 50 of the second ring-shaped element 48 and are accommodated by an outer surface of the first ring-shaped element 46. This has the aspect that the support arms are secured and held in place, i.e. a relative arrangement of the support arms is maintained during biasing of the outer portion 32 by the second biasing device 40. Thereby, an entangling and/or twisting may be avoided while collapsing the outer portion 32. In addition, the grooves 50 and the outer surface of the first ring-shaped portion 46 assist in radially inward biasing of the outer portion 32 along an outer surface of the inner sheath retaining the inner portion of the stent device in the collapsed state.

To maintain and facilitate the outer portion 32 into the collapsed state, a through-hole of the second cylindrical portion 44 has been accordingly dimensioned, such that the outer portion 32 may be accommodated within the second cylindrical portion 44 and be aligned along the inner portion and the inner sheath in a substantially concentric manner. Hence, upon longitudinal displacement, e.g. in the proximal direction, the outer portion 32 is biased radially inward and gradually received by the through-hole of the second cylindrical portion 44, wherein the end face of the cylindrical portion facing the outer portion 32 may engage the outer portion and facilitate the radially inward biasing.

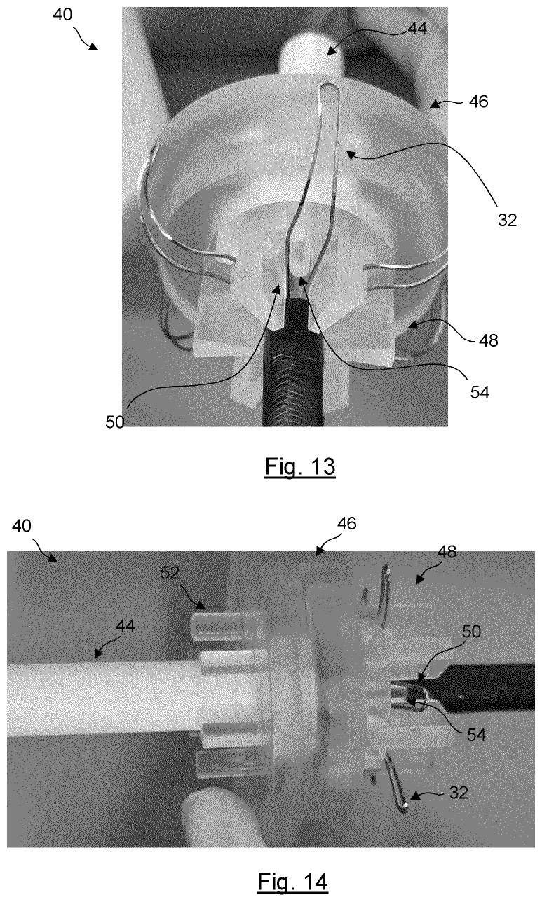

As shown in FIGS. 14 and 15, the first pins 54 may engage respective support arms of the outer portion 32 and facilitate that the support arms are retained secured within the grooves 50, when contact with the outer portion 32 is e.g. temporarily reduced during progression of the biasing and collapsing of the outer portion 32. As shown in FIG. 13, such securing function of the first pins 54 may not be required or at least not to the same extent when initiation the biasing of the outer portion 32, but may be desirable at a later stage of the collapsing process with the second biasing device 40, as shown e.g. in FIG. 15 just before the stent device has been brought in a final or predefined collapsed state.

In order to ensure that also the portion of the outer portion 32 being held by the first pins 54 may be biased and accommodated by the through-hole of the second cylindrical portion 44, the second pins 52 may be pressed so as to achieve a longitudinal displacement of the second ring-shaped element 48 relative to the first ring-shaped element 46, such that the first pins 54 no longer extend through the holes 60 or at least are no longer in contact with the corresponding portion of the outer portion 32. The second biasing device 40 may then be advanced over a remaining predefined portion of the outer portion 32.

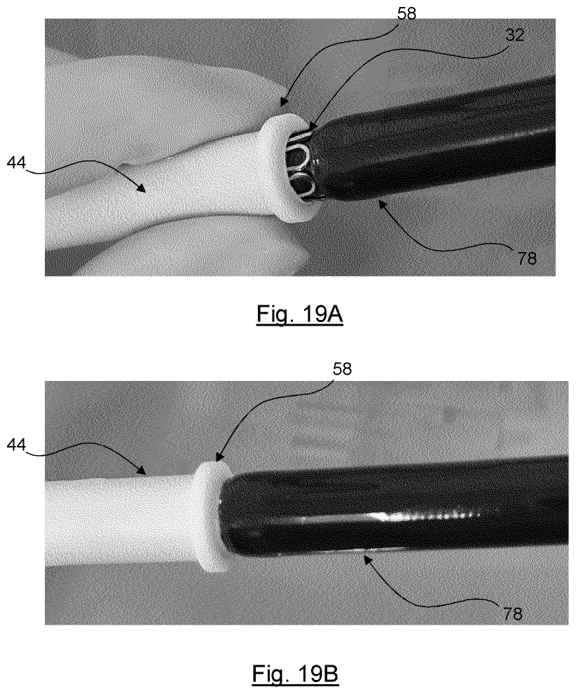

The ring-shaped securing device 42 may then be removed by decoupling the first ring-shaped element 46 and the second ring-shaped element 48, i.e. by a relative longitudinal displacement ensuring that the first pins 54 are no longer received by the holes 60 and the second pins 52 are no longer received by the holes 56. A corresponding state is depicted in FIG. 16, wherein only the second cylindrical portion 44 covers the collapsed portion of the outer portion 32. The remaining portion of the outer portion 32 may then be accommodated by the second cylindrical portion 44, such that a final or predefined collapsed state of the stent device, in embodiments, comprising a prosthetic heart valve, is achieved. Thereafter an outer sheath may be advanced over the hence collapsed outer portion by gradually removing the second cylindrical portion 44, e.g. in a distal direction, and successively advancing the outer sheath over the directly adjacent free portion of the outer portion 32, as shown in FIGS. 19A and 19B.

Thereby, a stent device comprising a prosthetic heart valve having an inner portion, e.g. an inner stent, and an outer portion 32, e.g. an outer support or anchoring frame, and which may particularly extend from a longitudinal end of the inner portion, may be collapsed in an efficient and space-saving manner, wherein the different radial extensions of the stent device portions and corresponding stiffnesses are accounted for. The inner portion and outer portion 32 may be particularly collapsed in a concentric arrangement in a two-step collapsing approach, wherein e.g. the relative positions of support arms of the outer portion 32 and between the outer portion and the inner portion may be maintained and excessive biasing forces or inadvertent deformations, which may otherwise be respectively lost or occur e.g. in a one-step collapsing approach, may be effectively avoided.

In FIG. 17, an alternative embodiment of the guiding device 10 is shown together with a corresponding sleeve component 24. According to the embodiment of FIG. 17, the retention surface 14 also has a radially flared or disc shape. However, neither the retention surface 14 nor the support surface 20 comprises the curvature 30 depicted in the embodiment according to FIG. 3. Instead, the retention surface 14 and the support surface 20 radially extend substantially in a straight or flat manner. To accommodate the retention surface 14, the support surface 20 according to the present example comprises a recess 64, which is formed by an inner wall that is shown to define a rim portion partially supporting the retention surface 14. Thereby, lateral and proximal movement of the guiding device 10 relative to the sleeve component 24 may be avoided.

In order to secure the guiding device 10 to the sleeve component 24 in a distal and/or circumferential direction, the retention surface 14 furthermore comprises a circumferential groove 62, which is surrounded by the inner wall of the support surface 20 in the assembled state. The circumferential groove 62 may be releasably engaged by a pin 66 or another suitable securing element via a through-hole 68 in the wall of the supporting surface 20, thereby limiting movement of the guiding device 10 in the distal direction, in embodiments, also in the circumferential direction.

FIG. 18 shows details of protrusions of a slidable component 28 of the first biasing device 22, wherein the protrusions are formed as wedge-like elements 70. In the assembled state, the rotatable component 26 is arranged around a top or distal end portion of the slidable component 28. In order to engage the slidable component 28, which is received within the sleeve component (not shown in this Figure), the wedge-like elements 70 are inserted through the rotatable component 26 via respective holes 74 and are secured to a corresponding outer thread 76 of the slidable component 28 using screws 72 or corresponding fixation means. In the fixed state of the wedge-like elements 70 to the slidable component 28, the wedge-like elements 70, do not extend through the holes 74, such that they do not impair rotation of the rotatable component 26.

To engage the wedge-like elements 70 and thus the slidable component 28, the rotatable component 26 may e.g. define an inner rim portion having a smaller radial extension than an inner diameter of the holes 74. Thereby, upon rotation of the rotatable component 26, said rim portion may longitudinally displace the wedge-like elements 70 within the slits of the sleeve component in a proximal direction, such that the slidable component is longitudinally advanced out of the sleeve component 24 in the corresponding direction, thereby facilitating a radial biasing and collapsing of the stent device received in the cavity 16 of the guiding device 10.

In FIGS. 19A and 19B the stent device is shown in a predefined or fully collapsed state, wherein the second outer portion or outer frame 32 of the stent device has been collapsed around the first inner portion and the inner sheath. In said collapsed state, an outer sheath 78 may be successively advanced over the outer frame 32, i.e. directly adjacent to the stop 58, while successively removing the second cylindrical portion 44, i.e. in a distal direction. The predefined collapsed stent device obtained thank to the two collapsing steps comprises: (a) a first inner portion of the stent device being collapsed in the inner sheath and (b) a second outer portion (or outer frame 32) of the stent device being collapsed in the outer sheath around the inner sheath (and thus first inner portion). This is particularly desirable during delivery of the stent device in a patient to be treated at a targeted site, for example to replace a heart valve (e.g. aortic, mitral or tricuspid heart valve) as this allows to first deploy the outer portion of the stent device (e.g. support or anchoring frame), to adjust its positioning within the targeted site (e.g. native mitral or tricuspid valve annulus) and finally to deploy the inner portion of the stent device (e.g. containing a prosthetic valve, e.g. a prosthetic tricuspid valve) within the targeted site.

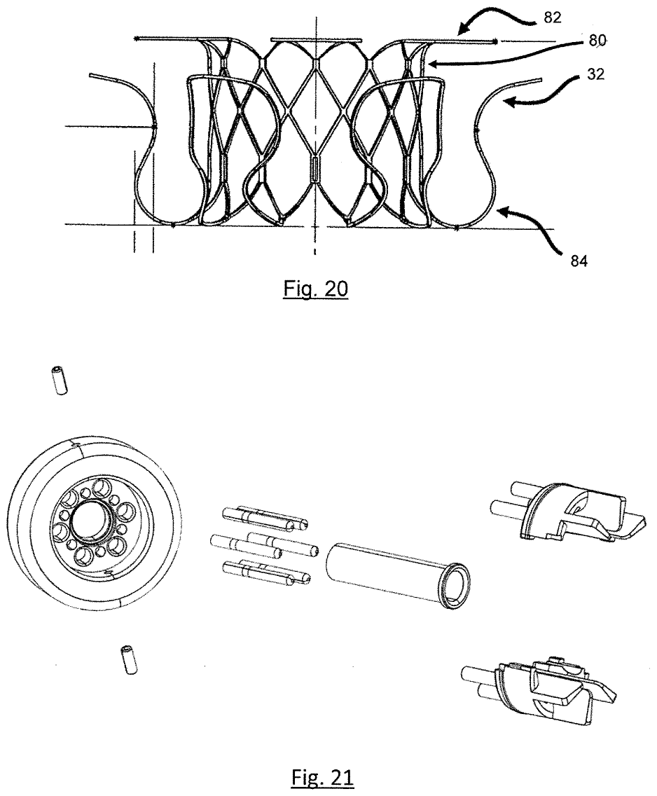

FIG. 20 shows an exemplary collapsible stent device compatible with the loading system according to the present disclosure. Accordingly an inner portion 80 of the stent device is surrounded by an outer frame 32. Within the inner portion, a prosthetic valve, e.g. a prosthetic tricuspid valve may be securely held while the outer frame 32 may facilitate an anchoring of the stent device, e.g. within the native tricuspid valve annulus of a patient to be treated. When collapsing the stent device, a proximal end 82 may be inserted into the cavity 16 of the guiding device 10 and subsequently collapsed in a first step, resulting in an intermediate collapsed state. Then, in a second step, the outer frame 32 may be collapsed around the inner portion 80 in a second step, wherein the outer frame 32 is inserted into the second cylindrical portion 44 from the distal end 84 towards the proximal end 82.

As described above, the terms “distal” and “proximal” in this regard are to be understood in the context of their common use when applying catheters, The longitudinal end being closest to the patient, e.g. a nose cone on a guide wire, is considered as a “distal end” and a longitudinal end being closest to a surgeon, e.g. a steering knob or handle, is considered as a “proximal end”. In embodiments, in the implanted and deployed state, however, the proximal end 82 forms an atrial portion of the stent device while the distal end 84 forms a ventricular portion, such that the terms “proximal” and “distal” may have a different meaning, i.e. be inverted, as seen from direction of blood flow.

It will be obvious for a person skilled in the art that these embodiments and items only depict examples of a plurality of possibilities. Hence, the embodiments shown here should not be understood to form a limitation of these features and configurations. Any possible combination and configuration of the described features can be chosen according to the scope of the present disclosure.

### LIST OF REFERENCE NUMERALS

10 Guiding device
12 First cylindrical portion
14 Retention surface
16 Cavity
18 Opening
20 Support surface
22 First biasing device
24 Sleeve component
26 Rotatable component
28 Slidable component
30 Curvature
32 Outer portion or outer frame
34 Coupling mechanism
36 Coupling element
38 Slit
40 Second biasing device
42 Ring-shaped securing device
44 Second cylindrical portion
46 First ring-shaped element
48 Second ring-shaped element
50 Groove
52 Second pin
54 First pin
56 Hole
57 Recess
58 Stop
60 Hole
62 Circumferential groove
64 Recess
66 Pin
68 (Through) Hole
70 Wedge-like element
72 Screw
74 Hole
76 Thread
78 Outer sheath
80 Inner portion
82 Proximal end
84 Distal end

1. A loading system for collapsing a stent device for a prosthetic heart valve into a predefined state, comprising
a guiding device (10) comprising:
an open cavity (16) defined by a wall and configured to receive a longitudinal end portion of the stent device, wherein a diameter of the cavity (16) increases from a longitudinal end of the cavity (16) defining an opening (18) to an opposing longitudinal end of the cavity (16), and
a first cylindrical portion (12) defining a through-hole having a predefined inner diameter and extending from said opening (18) in a longitudinal direction away from the cavity (16),
a first biasing device (22) configured to accommodate the first cylindrical portion (12) and comprising a coupling mechanism (34) for coupling the first biasing device (22) to the stent device received in the cavity (16) and a biasing mechanism, wherein the biasing mechanism is configured to bias a portion of the stent device into the through-hole via the cavity (16) and the opening (18) in the coupled state of the stent device.

2. A loading system for collapsing a stent device for a prosthetic heart valve into a predefined state, comprising
a guiding device (10) comprising:
an open cavity (16) defined by a wall and configured to receive a longitudinal end portion of the stent device, wherein a diameter of the cavity (16) increases from a longitudinal end of the cavity (16) defining an opening (18) to an opposing longitudinal end of the cavity (16), and
a first cylindrical portion (12) defining a through-hole having a predefined inner diameter and extending from said opening (18) in a longitudinal direction away from the cavity (16),
a first biasing device (22) configured to accommodate the first cylindrical portion (12) and comprising:
a slidable component (28) comprising a coupling mechanism (34) for coupling the first biasing device (22) to the stent device received in the cavity (16),
a rotatable component (26) engageable with the slidable component so as to provide only a longitudinal displacement of the slidable component (28) upon rotation of the rotatable component (26) and
a sleeve component (24) so as the slidable component (28) is slidably arranged within the sleeve component (24),
wherein the first biasing device is configured to bias a portion of the stent device into the through-hole via the cavity (16) and the opening (18) in the coupled state of the stent device.

3. The loading system according to claim 1 or 2, wherein the wall of the cavity (16) defines an inner guiding surface and/or inner support surface for the stent device configured to facilitate the biasing of the stent device in the coupled state.

4. The loading system according to claim 1, 2 or 3, wherein the diameter of the cavity (16) gradually increases from the opening (18) towards the opposing longitudinal end of the cavity (16).

5. The loading system according to any of the preceding claims, wherein the cavity (16) has a conical or parabolic shape in a longitudinal section of the cavity (16) and/or has a rotationally symmetrical shape along a longitudinal axis defined by the cavity (16).

6. The loading system according to any of the preceding claims, wherein an inner surface of the wall defining the cavity (16) and an outer surface of the wall are similarly shaped and/or essentially extend in parallel in a longitudinal section of the cavity (16).

7. The loading system according to any of the preceding claims, wherein the diameter of the opening (18) and the inner diameter of the through-hole are equal.

8. The loading system according to any of the preceding claims, wherein a length of the first cylindrical portion (12) in the longitudinal direction is configured to only accommodate a first inner portion of the stent device.

9. The loading system according to any of the preceding claims, wherein the guiding device (10) and the first biasing device (22) are configured to bias the stent device into an intermediate collapsed state.

10. The loading system according to any of the preceding claims, wherein the guiding device (10) comprises a retention surface (14) configured to engage a surface of the first biasing device (22) and to maintain a position of the guiding device (10) relative to a portion of the first biasing device (22).

11. The loading system according to claim 10, wherein the retention surface (14) is arranged at the level of the opening (18) and/or the first cylindrical portion (12) and/or wherein the retention surface (14) is formed as a radially extending flange portion.

12. The loading system according to any of the preceding claims, wherein the first biasing device (22) is configured to provide a longitudinal biasing force to the stent device.

13. The loading system according to claim 1 or any of the preceding claims as far as dependent on claim 1, wherein the first biasing device (22) comprises a slidable component (28) comprising the coupling mechanism (34) and a rotatable component (26) engageable with the slidable component (28) so as to provide only a longitudinal displacement of the slidable component (28) upon rotation of the rotatable component (26).

14. The loading system according to claim 13 or claim 2 or any of the preceding claims as far as being dependent on claim 2, wherein the first biasing device (22) further comprises a sleeve component (24), wherein the slidable component (28) is slidably arranged within the sleeve component (24) and preferably the rotatable component (26) is arranged at least partially around an outer surface of the sleeve component (24).

15. The loading system according to claim 14 or claim 2 or any of the preceding claims as far as being dependent on claim 2, wherein the sleeve component (24) comprises at least one longitudinal slit (38) and wherein the slidable component (28) and the rotatable component (26) are mechanically couplable or engageable with each other via the at least one longitudinal slit (38).

16. The loading system according to claim 15, wherein the slidable component (28) comprises at least one radially extending protrusion and wherein each longitudinal slit (38) is dimensioned to accommodate a respective protrusion, wherein the rotatable component (26) is configured to engage the at least one protrusion at an outer surface of the sleeve component (24).

17. The loading system according to any of the preceding claims, wherein the sleeve component (24) comprises a support surface (20) configured to provide a mechanical coupling with the guiding device (10) in the assembled state.

18. The loading system according to claim 17, wherein the support surface (20) is configured to engage a retention surface (14) of the guiding device (10).

19. The loading system according to claim 18, wherein the support surface (20) comprises at least one curvature (30) in a longitudinal section configured to receive and accommodate the retention surface (14) or wherein the support surface (20) defines a recess (64) accommodating and surrounding the retention surface (14).

20. The loading system according to any of claims 17 to 19, wherein the support surface (20) is formed as an essentially radially extending flange.

21. The loading system according to any of claims 17 to 20, wherein the mechanical coupling is provided in a form-fitting manner in the assembled state and the coupled state of the stent device and/or wherein the retention surface (14) defines a circumferential groove (62) being releasably engageable by a securing element via a through-hole (68) in a wall of the supporting surface (20).

22. The loading system according to any of claims 13 to 21 or any of claims 2 to 21, as far as being dependent on claim 2 wherein the slidable component (28) is formed as a hollow cylinder defining a longitudinal through-hole.

23. The loading system according to any of claims 14 to 22 or of claims 2 to 22 as far as being dependent on claim 2 wherein the sleeve component (24) is configured to receive and accommodate the first cylindrical portion (12) of the guiding device (10) and/or to bias at least the first cylindrical portion (12) and the slidable component (28) in a concentric arrangement in the assembled state.

24. The loading system according to any of the preceding claims, wherein the coupling mechanism (34) comprises at least one retention groove, cut-out, and/or recess configured to secure a coupling element (36) of the stent device in a form-fitting manner at least in the longitudinal direction away from the cavity (16).

25. The loading system according to any of the preceding claims comprising a second biasing device (40) configured to receive an opposing longitudinal end of the stent device and/or to receive a portion of the stent device in an intermediate collapsed state.

26. The loading system according to claim 25, wherein the second biasing device (40) is configured to engage a second outer portion (32) of the stent device.

27. The loading system according to claim 25 or 26, wherein the second biasing device (40) is configured to bias a portion being in an expanded state around a portion of the stent device being in a collapsed state.

28. The loading system according to any of claims 25 to 27, wherein the second biasing device (40) comprises a second cylindrical portion (44) defining a through-hole having a predefined inner diameter and being configured to radially bias a portion of the stent device and accommodate the portion of the stent device within the through-hole upon longitudinal displacement of the second cylindrical portion (44) over and relative to the portion of the stent device.

29. The loading system according to claim 28, wherein the inner diameter of the second cylindrical portion (44) is larger than or essentially corresponds to an outer diameter of the first cylindrical portion (12).

30. The loading system according to claim 28 or 29, wherein the second biasing device (40) comprises a ring-shaped securing device (42) being releasably couplable to the second cylindrical portion (44) and configured to engage a stop (58) formed by the second cylindrical portion (44) only in one longitudinal direction.

31. The loading system according to claim 30, wherein the stop (58) is formed as a radially extending flare.

32. The loading system according to claim 30 or 31, wherein the ring-shaped securing device (42) comprises two or more grooves (50) at a longitudinal end face engaging the stent device, wherein the grooves (50) are spaced-apart from each other in a circumferential direction and wherein each groove (50) is configured to secure a respective support arm of the stent device in a circumferential direction.

33. The loading system according to claim 32, wherein the grooves (50) extend in a longitudinal and radial direction.

34. The loading system according to claim 32 or 33, wherein the grooves (50) are formed to engage and bias the respective support arm in a longitudinal and/or radial direction upon longitudinal displacement of the ring-shaped securing device (42) relative to the portion of the stent device.

35. The loading system according to any of claims 32 to 34, wherein each groove (50) comprises a respective first pin (54) configured to secure the respective support arm of the stent device in the circumferential direction.

36. The loading system according to any of claims 30 to 35, wherein the ring-shaped securing device (42) is formed by a first ring-shaped element (46) and a second ring-shaped element (48) that are releasably couplable.

37. The loading system according to claim 36, wherein the first and second ring-shaped elements (46, 48) are decouplable by a relative displacement in the longitudinal direction.

38. The loading system according to claim 36 or 37, wherein the second ring-shaped element (48) comprises second pins (52) dimensioned and arranged to extend through and beyond holes (56) in the first ring-shaped element (46) in the coupled state of the first and second ring-shaped elements (46, 48).

39. The loading system according to claim 38, wherein the second pins (52) are circumferentially spaced apart and configured to rotationally secure the first and second ring-shaped elements (46, 48) relative to each other in said coupled state.

40. The loading system according to any of claims 36 to 39, wherein the second ring-shaped element (48) comprises holes (60) dimensioned and arranged for receiving and accommodating respective first pins (54) of the first ring-shaped element (46).

41. The loading system according to any of claims 36 to 40, wherein the second ring-shaped element (48) is at least partially accommodated by a recess (57) of the first ring-shaped element (46) in the coupled state.

42. The loading system according to any of claims 36 to 41, wherein the second ring-shaped element (48) is formed of equally formed parts that are releasably couplable to each other.

43. The loading system according to any of the preceding claims, configured for collapsing a stent device comprising a prosthetic heart valve, preferably a tricuspid or mitral heart valve.
